(19) United States
(12) Patent Application Publication
(10) Pub. No.: US 2012/0130164 A1
(43) Pub. Date: May 24, 2012

(54) MAGNETIC BASED DEVICE FOR RETRIEVING A MISPLACED ARTICLE

(75) Inventors: Michael Palese, New York, NY (US); Daniel Gainsburg, Chappaqua, NY (US)

(73) Assignee: Mount Sinai School of Medicine, New York, NY (US)

(21) Appl. No.: 13/303,050

(22) Filed: Nov. 22, 2011

Related U.S. Application Data
(60) Provisional application No. 61/537,358, filed on Sep. 21, 2011.
(60) Provisional application No. 61/416,814, filed on Nov. 24, 2010.

(51) Int. Cl. A61B 1/00

(52) U.S. Cl. 600/104

(57) ABSTRACT

A device for locating and retrieving a misplaced foreign body that has metallic characteristics includes a shaft connected to a handle and a retrieval tool that is coupled to the shaft and moves between a deployed position and collapsed position. The tool has a magnetic element that generates a magnetic field, wherein the magnetic field extends over a greater area in the deployed position and is of sufficient strength to attract the foreign body such that it is held in contact with the tool.

### CROSS REFERENCE TO RELATED APPLICATION

The present application claims the benefit of U.S. patent application Ser. No. 61/416,814, filed Nov. 24, 2010, and U.S. patent application Ser. No. 61/537,358, filed Sep. 21, 2011, each of which is hereby incorporated by reference in its entirety.

### TECHNICAL FIELD

The present invention relates to medical equipment, and in particular, to a device or probe for retrieving an article (foreign body) that is attracted to a magnetic field and has been misplaced during a surgical procedure, such as during a minimally invasive surgical procedure (e.g., a laparoscopic, robotic, endoscopic or arthroscopic procedure).

### BACKGROUND

As technology advances, the manner in which surgical procedures are conducted and the instruments/tools that are used have greatly changed and the field of minimally invasive surgery has spawned many types of surgical procedures including laparoscopic, robotic, endoscopic and arthroscopic surgery. Laparoscopy (laparoscopic surgery) is an operation performed in (but not limited to) the chest, abdomen, pelvis, or retroperitoeneum through small incisions (usually 0.5-1.5 cm) with the aid of a camera. It can either be used to inspect and diagnose a condition or to perform surgery. The laparoscope allows doctors to perform both minor and complex surgeries with a few small incisions. There are a number of advantages to the patient with laparoscopic surgery versus an open procedure. These include reduced pain, decreased blood loss, and faster recovery time. Robotic surgery is the latest advance in minimally invasive surgery which is similar to laparoscopic surgery in that surgery is performed through (but not limited to) small incisions in the chest, abdomen, pelvis or retroperitoneum but uses a robotic system to perform surgery.

Endosocpic surgery is any surgery through a scope or instrument into a body cavity such as endoscopy in the upper gastro-intestinal tract or cystoscopy in the lower genitor-urinary tract. This is a minimally invasive procedure that allows for the examination and treatment of tissues, organs, etc with a scope that has a camera and a working channel.

Arthroscopy (also called arthroscopic surgery) is a minimally invasive surgical procedure in which an examination and sometimes treatment of damage of the interior of a joint is performed using an arthroscope, a type of endoscope that is inserted into the joint through a small incision. Arthroscopic procedures can be performed either to evaluate or to treat many orthopaedic conditions including torn floating cartilage, torn surface cartilage, ACL reconstruction, and trimming damaged cartilage.

Despite guidelines to improve practice, the occurrence of retained items during or after surgery remains problematic. Perioperative nurses perform surgical counts of items to be used during a procedure before surgery begins and at specified times during and at the end of surgery to ensure that items are not left in a patient unintentionally. In its statement on the prevention of retained foreign bodies after surgery, the American College of Surgeons recommends consistent application and adherence to standardized counting procedures and performance of methodical wound exploration before closure of the surgical site.

All surgical sponges and instruments utilized during any surgical procedure must be accounted for at the end of the operation to ensure no foreign body is inadvertently left inside the patient. Individual institutions establish their own counting policies based on guidelines from the Association of Peri-operative Registered Nurses. Retained surgical foreign bodies are most frequently reported in the chest, abdomen, pelvis or vagina.

Current practice regarding the misplacement of intra abdominal foreign bodies involves meticulous and systematic examination of the peritoneal cavity with some sources recommending that the ability to have visual and tactile recognition of the foreign body being particularly important. The potential for a needle stick injury is amplified by this technique. If the object is not found, the next step involves getting a radiographic imaging study and the patient remains in the operating room until the film is reviewed by a radiologist. However, in many institutions, the act of ordering an x-ray is viewed cautiously and as an act of last resort since most times when an x-ray is ordered, an internal report must be generated as to why the x-ray is needed. This exposes all those involved to at least a note in one's professional record or institution and possibly to reprimand since the cause of the need for the x-ray is the misplacement of a foreign body.

While there has been suggestions and recommendations including providing x-ray detectable (radiographic) packs, gauze, and needles, there is a need for an improved device that is configured to be used with a port or instrument that is used in a surgical procedure (e.g., a minimally invasive procedure) and configured to retrieve a misplaced foreign body that is attracted to a magnetic field.

### SUMMARY

In one embodiment, a device for locating and retrieving a misplaced foreign body that has metallic characteristics includes a shaft connected to a handle and a retrieval tool that is coupled to the shaft and moves between a deployed position and collapsed position. The tool has a magnetic element that generates a magnetic field, wherein the magnetic field extends over a greater area in the deployed position and is of sufficient strength to attract the foreign body such that it is held in contact with the tool.

In another embodiment, a device for locating and retrieving a misplaced foreign body that has metallic characteristics includes a shaft connected to a handle and an electro-magnetic element that is disposed about the shaft and is configured to generate a magnetic field when electric current passes therethrough. The shaft and electro-magnetic element have a width of about 20 mm or less to allow the device to be used in ports associated with minimally invasive surgery.

A method for locating and retrieving a misplaced metal foreign body within a cavity of the body including the steps of: (1) inserting a port through an incision made in the body, the port providing a conduit to the cavity; (2) inserting a device through the conduit of the port for locating and retrieving the misplaced metal foreign body, the device including a shaft connected to a handle and a retrieval tool that is coupled to the shaft and moves between a deployed position and collapsed position, the tool having a magnetic element that generates a magnetic field; (3) deploying the retrieval tool after the retrieval tool has cleared the port and is within the cavity, wherein the magnetic field extends over a greater area in the deployed position; and (4) moving the retrieval tool within the cavity until the misplaced foreign body is under the influence of the magnetic field and is drawn into contact and held against the magnetic element of the retrieval tool.

These and other aspects, features and advantages shall be apparent from the accompanying Drawings and description of certain embodiments of the invention.

### BRIEF DESCRIPTION OF THE DRAWINGS

FIG. 1 is a perspective side view of a device for retrieving an article that has been misplaced during a surgical procedure showing a tool in a retracted position;

FIG. 2 is perspective side view of the device of FIG. 1 with the tool in the fully extended position;

FIG. 3 is an exploded perspective view of the device of FIG. 1 showing inner working components according to a first embodiment;

FIG. 4 is a cross-sectional view taken along the line 4-4 of FIG. 2;

FIG. 5 is a cross-sectional view taken along the line 5-5 of FIG. 1;

FIG. 6 is a cross-sectional view of the tool of FIG. 1 in a partially extended position;

FIG. 7 is a cross-sectional view of the tool of FIG. 1 in a fully extended position;

FIG. 8 is a close-up of a portion of the tool of the device showing magnets disposed within recesses formed in the tool;

FIG. 9 is a close-up of a portion of the tool of the device showing magnets disposed within holes formed in the tool;

FIG. 10 is a close-up of a portion of the tool of the device showing magnet strips disposed longitudinally along a length of the tool;

FIG. 11 is side perspective view of the tool of the device of the FIG. 8 being inserted through a trocar and delivered to a surgical site for retrieving an article that has been misplaced during a minimally invasive surgical procedure;

FIG. 12 is a side perspective view of a tool according to another embodiment being inserted through a trocar and delivered to a surgical site for retrieving an article that has been misplaced during a minimally invasive surgical procedure;

FIG. 13 is a perspective side view of another device for retrieving an article that has been misplaced during a surgical procedure showing a tool in a retracted position;

FIG. 14 is perspective side view of the device of FIG. 13 with the tool in the fully extended position;

FIG. 15 is a cross-sectional view taken along the line 15-15 of FIG. 14;

FIG. 16 is an exploded perspective view of a tool according to one embodiment;

FIG. 17 is top view of the tool of FIG. 16;

FIG. 18 is a perspective view of a tool according to another embodiment;

FIG. 19 is a perspective view of a tool according to yet another embodiment;

FIG. 20 is a perspective view of a tool according to another embodiment with magnetic splines (fingers) in an extended (deployed) state;

FIG. 21 is cross-sectional view taken along the line 21-21 of FIG. 20;

FIG. 22 is a cross-sectional view showing the magnetic splines in a retracted position;

FIG. 23 is a perspective view of an electro-magnetic device for retrieving an article that has been misplaced during a surgical procedure;

FIG. 24 is a perspective view of another electro-magnetic device for retrieving an article that has been misplaced during a surgical procedure;

FIG. 25 is a perspective view of another magnetic device for retrieving an article that has been misplaced during a surgical procedure, with the device being in the closed or retracted position;

FIG. 26 is a perspective view of the device of FIG. 25 in an opened or extended position;

FIG. 27 is a close-up perspective view of the magnetic element at the distal end of the device showing the magnetic element in a number of different pivoted positions;

FIG. 28 is a close-up perspective view of the magnetic element with an outer sheath being removed;

FIG. 29 is another close-up perspective view of the magnetic element with the outer sheath being removed;

FIG. 30 is a close-up perspective view of a proximal end of the device with the device in an opened (extended) position; and

FIG. 31 is a close-up perspective view of the proximal end, with the device being shown in the closed (retracted) position.

### DETAILED DESCRIPTION OF CERTAIN EMBODIMENTS OF THE INVENTION

In accordance with the present invention, a device is provided that is configured to be used in minimally invasive surgical procedures and is constructed to retrieve misplaced foreign bodies to prevent the retention of foreign bodies in the patient after surgery.

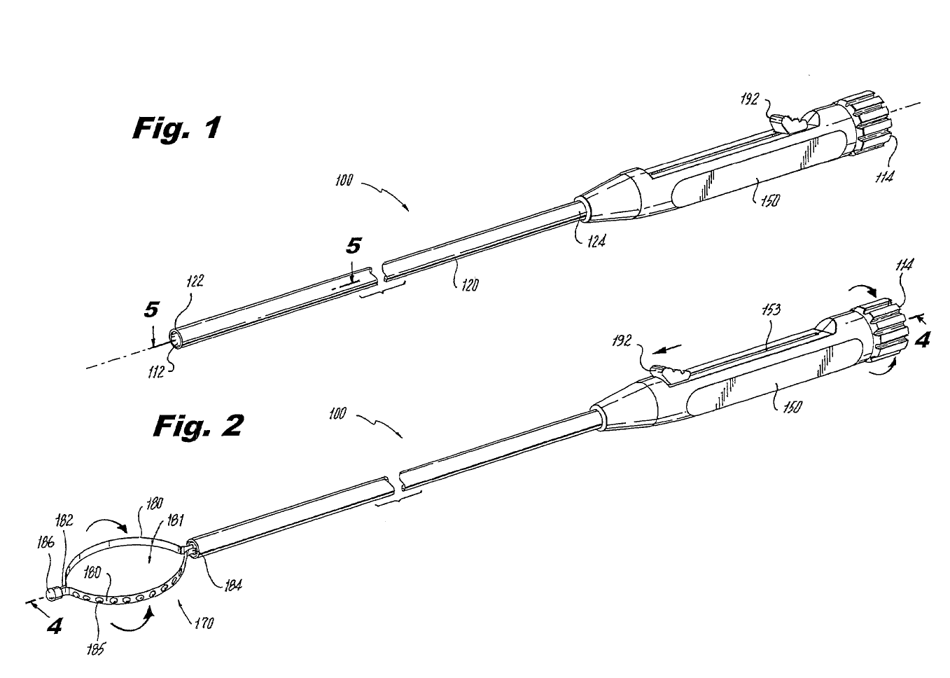

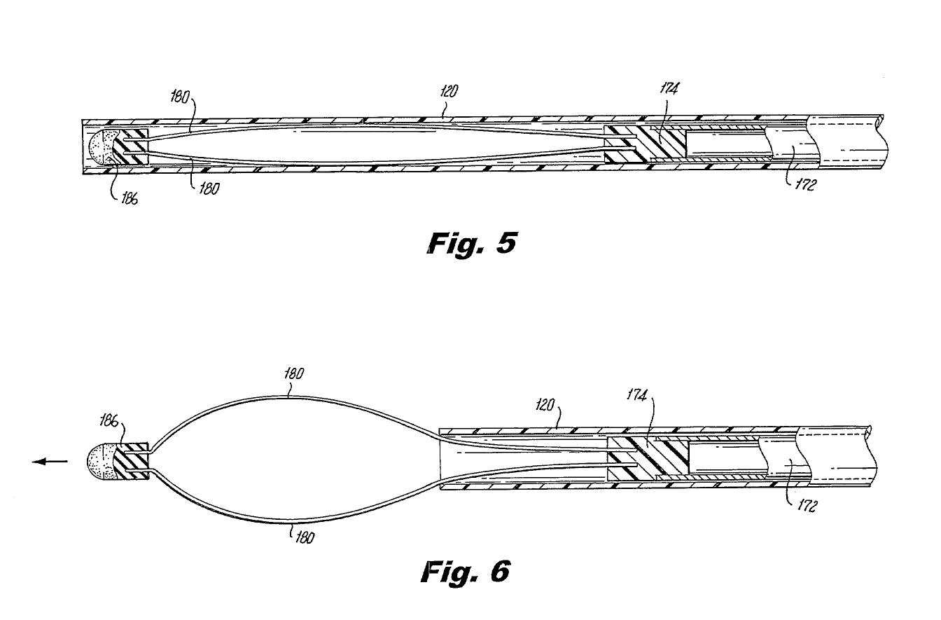

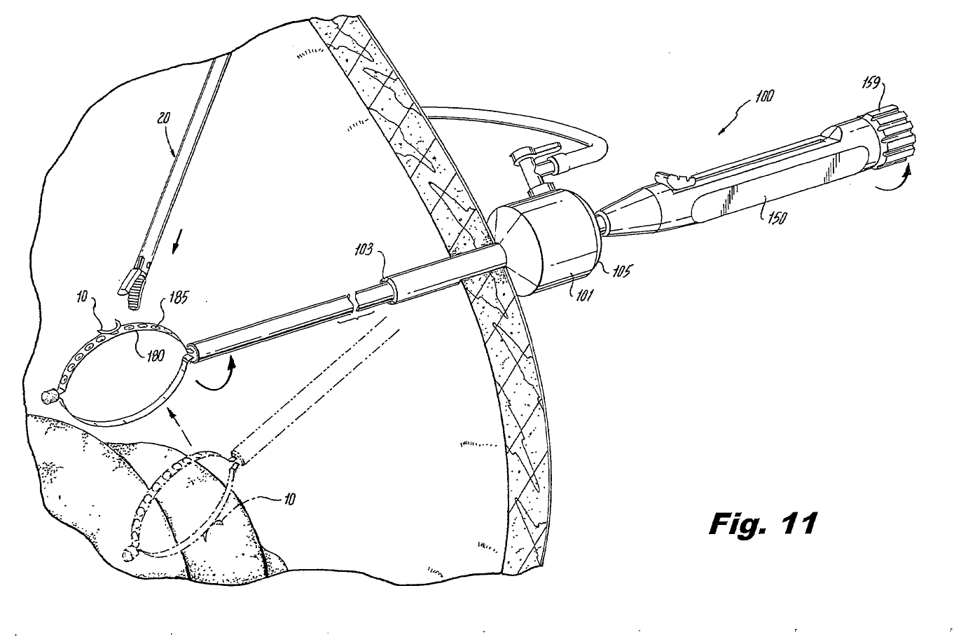

FIGS. 1-7 illustrate a device 100 according to a first embodiment for insertion into a conventional trocar/port 101 (FIG. 11) that provides access to the inside of the patient during minimally invasive surgery and for locating and retrieving a misplaced foreign body. As is known in the industry, a trocar is a medical instrument with a sharply pointed end, often three-sided, that is used inside a hollow cylinder (cannula) to introduce the trocar into blood vessels or body cavities. Trocars are also used to introduce ports in the abdomen, such as during laparoscopic surgery. Trocar sites are the small entry sites made though abdomen for the entry of surgical instruments. After a small incision is made in the skin, the trocar is the instrument inserted to penetrate the abdominal wall. Trocars of different sizes can create entry sites ranging from 5 to 20 mm in diameter. The diameter size depends on whether the removal of a specimen is anticipated and its size and other considerations, such as the size/age of the patient. In conventional laparoscopic surgery, 3-5 ports are typically used; however, single port laparoscopy (also known as “single port surgery”) is the newest frontier in laparoscopic surgery. Laparoscopic surgery involves inflating the abdomen with an inert gas (CO₂) and performing an operation seen through a thin camera tube along with several long thin instruments inserted through separate “ports” or trocars. Single port laparoscopy uses just one port buried in the belly button to accommodate both the instruments and the camera. This would eliminate the use of up to 3 to 5 separate trocars for the performance of typical laparoscopic procedures and would potentially leave the patient with no visible scars.

In order to be an effective tool in locating and retrieving misplaced foreign bodies and have wide surgical applicability, the device of the present invention is constructed (e.g., sized and shaped) so that it can be both inserted and removed through conventional medical equipment, such as the above-described trocars/ports that are used in minimally invasive surgeries.

FIG. 11 illustrates a trocar/port 101 that is inserted into the patient to provide access to the surgical site, such as a body cavity (e.g., abdomen). The trocar 101 has a first end 103 and an opposite end 105 that remains outside of the patient. A bore is formed through the trocar 101 that permits instruments to be passed therethrough to the surgical site, such as a body cavity.

It will be appreciated that the misplaced foreign bodies can include but are not limited to needles, sponges, gauze and other surgical instruments that are used during the surgical procedure. Many of these items have a metallic nature and in particular, needles and surgical instruments that are used in minimally invasive surgery have a metallic nature since at least a portion of the device is formed of a metal. The conventional radioscopic technique of locating misplaced foreign bodies takes advantage of this fact since the misplaced foreign body appears in the x-ray and its location can thus be determined.

As shown in FIGS. 1-7, the device 100 has an elongated body that has a first end 112 and an opposing second end 114. The first end 112 is in the form of a distal end, while the second end 114 represents a proximal end of the device. The device 100 generally can be thought of as including a shaft 120 that extends to and terminates at the distal end 112 and a handle 150 that is located at the proximal end 114 of the device 100. The shaft 120 includes a free distal end 122 (which can be the first end 112) and an opposing proximal end 124 that is coupled to the handle 150. The shaft 120 is a hollow structure (e.g., a cannula) that has a working center bore that extends the length of the shaft 120 and is open at the distal end 122. The size and shape of the shaft 120 is complementary to the trocar bore since the shaft 120 must be able to be introduced into and pass the length of the trocar bore to the surgical site. Typically, both the trocar bore and the shaft 120 have a circular shape; however, other shapes are possible so long as the shapes are complementary.

The device 100 has a retrieval member or mechanism 170 for locating and retrieving a misplaced foreign body. The retrieval mechanism 170 is configured so that it moves between a fully extended (deployed) position and a fully retracted (collapsed) position and in some embodiments can also be placed into a position therebetween, namely, an intermediate, partially extended position. In the fully extended (deployed) position, an increase in exposed surface area of the mechanism 170 preferably results so as to create additional contact points that emit a magnetic field and can attract and retain the misplaced foreign body.

The retrieval mechanism 170 includes at least one magnetic element that attracts and retains the misplaced foreign body as described in detail herein. As is known, a magnet is a material or object that produces a magnetic field. The magnetic field is responsible for generating a force that pulls on other ferromagnetic materials like iron and attracts or repels other magnets. A permanent magnet is an object made from a material that is magnetized and creates its own persistent magnetic field. An everyday example is a refrigerator magnet used to hold notes on a refrigerator door.

The permanent magnets for use herein can be in the form of any conventional permanent magnet that is suitable for the intended application. In particular, permanent magnets can be form of ferromagnetic materials, including iron, nickel, cobalt, some alloys of rare earth metals, and some naturally occurring minerals such as lodestone. Ferromagnetic (and ferrimagnetic) materials are the only ones attracted to a magnet strongly enough to be commonly considered magnetic. Permanent magnets are made from “hard” ferromagnetic materials which are subjected to special processing in a powerful magnetic field during manufacture, to align their internal microcrystalline structure, making them very hard to demagnetize.

Another type of conventional magnet is an electromagnet that is made from a coil of wire which acts as a magnet when an electric current passes through it, but stops being a magnet when the current stops. Often an electromagnet is wrapped around a core of ferromagnetic material like steel, which enhances the magnetic field produced by the coil (the core acts like a bar (permanent) magnet). Thus, the coiling of the wire amplifies the wire's magnetic field. Typically, the more turns of wire results in a greater magnetic field being generated. The electric current can come from any number of different energy sources.

The retrieval mechanism 170 of the embodiment of FIGS. 1-7 is part of an inner elongated member 172, such as a tube, cannula, shaft or rod, that is received within the center bore of the shaft 120. For example, the retrieval mechanism 170 can be located at or near a distal end 174 of the elongated member 172. As mentioned above, the retrieval mechanism 170 includes a magnetic element and is configured so that it can be received and stored within the bore of the shaft 120. The illustrated retrieval mechanism 170 is formed along a length of the inner member 172 and includes at least two flexible splines 180 that are constructed so that they move between a fully collapsed position and a fully deployed position.

In one embodiment, the splines 180 thus represent a longitudinal section of the inner member 172 or can be located at the distal end 174 thereof and represents an extension of the inner member 172. In the illustrated embodiment, each spline 180 has a first end 182 that is attached to a tip member 186 and a second end 184 that is attached to a plug 187 or the like that is securely attached to the distal end 174 of the inner member 172. When two splines 180 are used, the splines 180 are located opposite one another. The splines 180 are designed so that that they naturally move between a fully deployed (extended) position and a fully collapsed (retracted) position. In particular, in a normal, rest position, the splines 180 assume the fully deployed position in which the splines 180 are bent along their lengths. As illustrated, the splines 180 can assume a convex form with the convex surfaces facing outward from one another. This results in a space 181 that is formed between the splines 180. When a force is applied to the splines 180 (e.g., an inwardly directed force), the splines 180 flex inward and this result in the splines 180 straightening out and assuming a more linear form. In other words, as the inward force is applied to the splines 180, the splines 180 move toward the fully collapsed position in which the splines 180 are more linear in shape and parallel and can be received within the bore of the shaft 120.

Each spline 180 is constructed so that it has magnetic properties to facilitate locating and retrieving a misplaced foreign body that itself has metallic properties and thus is attracted to a magnetic field. For example, the spline 180 can have one or more permanent magnets incorporated therein, with the permanent magnets being capable of being provided in different forms, such as strips, pads, etc. For example, each spline 180 can have at least one magnetic element 185 that is supported by a substrate and in particular, the spline 180 can be a layered structure with a flexible substrate 183 that is formed of a material that is outwardly biased and supports the at least one magnetic element 185.

Any number of different materials can be used to form the flexible substrate 183. For example, a shape memory alloy (SMA, smart metal, memory metal, memory alloy, etc.) is an alloy that “remembers” its original, cold forged shape. One of the commercial uses of shape memory alloy involves using the pseudo-elastic properties of the metal during the high temperature (austenitic) phase. Frames or supports can be made of a shape memory alloy as they can undergo large deformations in their high temperature state and then instantly revert back to their original shape when the stress is removed. This is the result of pseudoelasticity; the martensitic phase is generated by stressing the metal in the austenitic state and this martensite phase is capable of large strains. With the removal of the load, the martensite transforms back into the austenite phase and resumes its original shape. This property of the shape memory alloy allows the metal to be bent, twisted and pulled, before reforming its shape when released. Based on the foregoing, the splines 180 can be formed from a shape memory alloy that has characteristics that permit the intended function to be performed.

The splines 180 not only provide a flexing action to permit movement between the fully deployed position and the fully collapsed position but also acts as a carrier for the magnetic element(s) 185. The magnetic element 185 can be carried and supported by the spline substrate 183 by any number of different techniques including bonding or otherwise fastening the magnetic elements 185 to the substrate 183. It will be appreciated that the magnetic element(s) 185 can be countersunk within a recess formed in the substrate 183 so that the exposed surface of the spline 180 is relatively smooth (FIG. 8). This facilitates the splines 180 moving to the fully collapsed position where the splines 180 overlap one another.

The magnetic element 185 can take any number of different forms including but not limited to a strip, pad, etc. and it will be appreciated that the magnetic element 185 does not have to extend the entire length of the substrate 183 but rather can be provided only in a local area(s). For example, a plurality of magnetic elements 185, such as circular shaped permanent magnets, can be provided along the length of the spline 180 and positioned to face outwardly when the spline 180 is in the fully deployed position so as to generate an outwardly directed magnetic field.

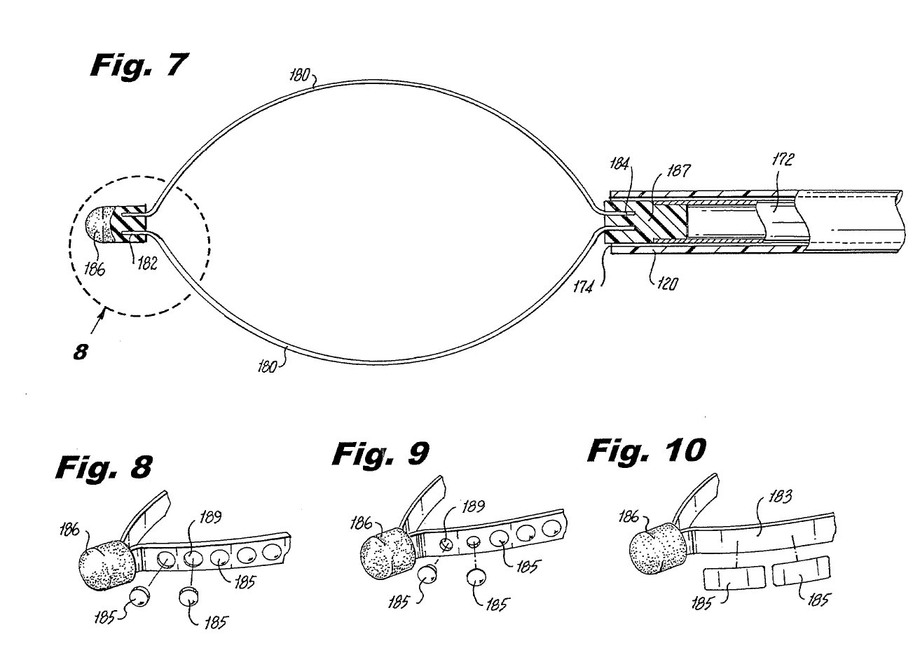

FIG. 8 shows the magnetic element 185 being received within a recess 189 formed in the spline 180 such that the outer surface of the magnetic element 185 can be flush relative to the outer surface of the spline 180. Each spline 180 can include a plurality of recesses 187 formed along the length of the spline 180. The recesses 187 do not pass completely through the body of the spline 180. FIG. 9 shows a different embodiment where the magnetic elements 185 are received within complementary openings (through-holes) 189 formed in the spline 180. The through-holes 189 pass completely through the body of the spline 180 and thus, the magnetic elements 185 are inserted into the through-holes 189 and adhered therein. FIG. 10 shows the magnetic elements in the form of strips 185 disposed along the exterior surface of the spline 180. The strips 185 can come in any shape and size.

It will be appreciated that the magnetic element(s) 185 are designed and positioned so that they do not interfere with the ability of the spline 180 to flex and move between the fully collapsed and fully deployed positions as described herein.

FIGS. 2 and 7 shows the splines 180 in the fully deployed position, while FIGS. 1 and 5 show the splines 180 in the fully collapsed position. The interaction and relative positions and/or movements between the inner member 172 and the shaft 120 result in the splines 180 moving between the fully deployed position and the fully retracted position. In the fully collapsed position, the splines 180 are positioned adjacent one another and are linearly arranged.

The device 100 can be of a disposable type and therefore after a single use, can be disposed of after the misplaced foreign body has been located and retrieved.

The handle 150 includes an actuator 190 for deploying the retrieval mechanism 170 and moving the retrieval mechanism 170 between the fully deployed and fully collapsed positions. The actuator 190 can come in any number of different forms and is designed so that the user can easily operate the device 100 with his or her hand. In the illustrated embodiment, the actuator 190 is in the form of a slide mechanism that includes a slider 192 that moves within a slot 153 that is formed in the handle 150. The slider 192 is operatively connected to the inner member 172 using a conventional mechanical attachment or linkage. For example, a pivotable link can be provided between the slider 192 and the inner member 172. The slider 192 is coupled to the inner member 172 such that linear movement of the slider 192 is translated into linear movement of the inner member 172 relative to the outer shaft 120 which in this embodiment is fixedly attached to the handle 150.

The length of the inner member 172 is such that when the slider 192 is moved to the most forward position which corresponds to the fully deployed position, the distal end 164 of the inner member 172 extends beyond the distal end of the outer shaft 120 so as to expose the retrieving mechanism 170. In the embodiment of FIG. 1, as the slider 192 is moved from the most rearward position, which corresponds to the fully collapsed (retracted) position, to the most forward position, the inner member 172 is moved forward within the bore of the shaft 120 resulting in the retrieving mechanism 170 (a tool) being exposed. Continued forward movement of the inner member 172 results in more of the retrieving mechanism 170 being exposed and due to the biasing (elastic) properties of the splines 180, the splines 180 flex radially outward away from one another. Once the slider 192 is moved into the most forward position, the splines 180 are at least substantially or completely exposed and have completely “sprung” open due to the material characteristics of the splines 180.

It will be appreciated that the actuator 190 can include a thumbwheel and be operated by rotation of the thumbwheel which causes movement of either the outer shaft 120 or the inner member 172. In addition, the handle can include a locking mechanism for locking the slider 192 in one of the positions.

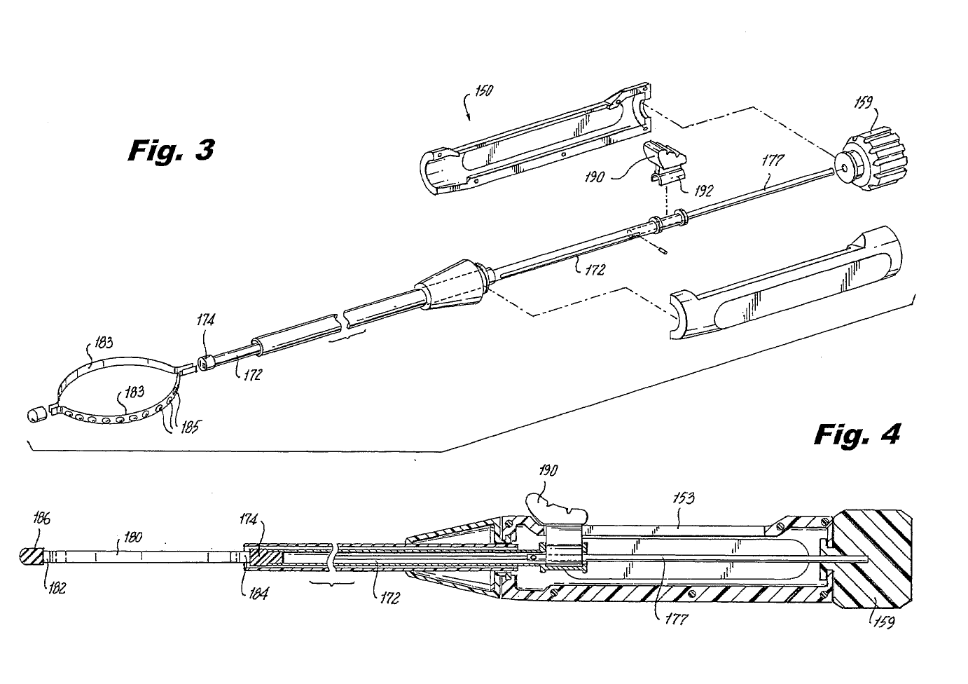

FIG. 3 also shows that that the retrieval mechanism 170 can be rotated. For example, the handle 150 can include a knob 159 at the proximal end. The knob 159 can be connected to the inner member 172 using a connector shaft 177 such that when the connector shaft 177 rotates, the inner member 172 likewise rotates, thereby causing rotation of the connected retrieval mechanism (tool 170).

It will also be appreciated that the retrieval mechanism 170 can be constructed so that additional other movements are possible. For example, in addition to being deployable and linearly movable and rotatable, the retrieval mechanism 170 can be constructed so that it can pivot in a different direction than the deployment direction.

As can be seen in the figures, once the retrieval mechanism 170 is fully deployed, the magnetic elements thereof are positioned for locating and retrieving a metal-based (metal-containing) misplaced foreign body due to an increased exposed surface area in the deployed position. Using cameras and the like, the device 100 is manipulated to cause the retrieval mechanism 170 to move within the surgical site and the magnetic field generated by the magnetic element 185 generates a force that attracts and draws a metal article, such as the metal-based misplaced foreign body, into contact with the magnetic element 185 when the metal article enters the magnetic field.

The strength of the magnetic field can be measured and the gauss (G) is the cgs unit of measurement of the magnetic field. It will be understood that the strength of the magnetic field is determined by a number of factors including the type of magnet since the material the permanent magnet is made from has a significant effect on the overall strength of the magnet. The magnetic field produced by deployment of the splines 180 is of sufficient strength to cause a metal-based misplaced foreign body to be drawn toward the splines 180 when the splines 180 are placed in a position proximate the foreign body during the search for the misplaced foreign body at the surgical site. As mentioned herein, once the misplaced foreign body is located, another tool can be used to remove the foreign body from the patient's body. In one embodiment the strength of the magnetic field is between 5 G to 100 G and in particular, 10 G to 90 G, etc. However, these values are merely exemplary and not limiting of the present invention since the strength of the desired magnetic field can depend upon the particular application and characteristics of the misplaced article.

The strength of the magnetic field can depend upon the particular application and can be selected depending upon different circumstances. The magnetic field is preferably of a strength that will attract a metallic foreign body that is within a prescribed radius from the foreign body. For example, the magnetic field strength can be selected so that a metallic foreign body that is within a distance of up to about 4 inches from the magnetic element is attracted towards the magnetic element for retrieval of the foreign body. It will be appreciated that this range is merely one exemplary embodiment and the magnetic field strength can be selected to achieve a different objective and attract an object that is at a different distance from the magnetic element; however, in general, the magnetic field should be selected so that metallic foreign bodies that are within a distance from the magnetic element are attracted to the magnetic element, wherein the distance is representative of a distance that the foreign body may travel from the surgical site when misplaced. In other words, the magnetic field strength should be selected so that metallic foreign bodies that are commonly used in the surgical procedure are attracted and retrieved when they migrate away from the surgical entry point but remain within a zone of travel that would be expected when the metallic foreign body is misplaced.

To return the tool (retrieval mechanism 170) to the fully collapsed position, the slider 192 is moved in the opposite direction (rearwardly) to cause the inner member 172 to move rearwardly within the shaft 120, thereby drawing the splines 180 toward the outer shaft 120. As the open splines 180 are drawn toward the shaft 120, the outer shaft 120 contacts and applies a force against the splines 180 to cause inward flexing of the splines 180. As the splines 180 are continuously drawn into the bore of the outer shaft 120, the splines 180 collapse into the fully collapsed position once the splines 180 are entirely contained within the shaft 120.

When used in minimally invasive surgery where a metal-based foreign body has been misplaced, the device 100 is inserted into the bore of the trocar/port 101 in the fully collapsed position and once at least the distal end of the device 100 clears the trocar/port and is located at the surgical site, the actuator 190 can be manipulated to cause deployment of the retrieving mechanism 170. Once the retrieving mechanism 170 is deployed and the misplaced foreign body is located and retained, another tool (e.g., a gripper) can be delivered to the surgical site through another port and brought into position to engage and remove the foreign body through the other port.

It will also be understood that instead of placing permanent magnets along the splines 180, each spline 180 can function as or include an electro-magnetic element such as the ones described herein. For example, a coil of wire can be disposed about each spline 180 and be operatively connected to an energy source, thereby providing an actuatable electro-magnet when current is passed therethrough. In this embodiment, after outward deployment of the splines 180, a current can be passed through the coil of wire to generate a magnetic field. The characteristics, including strength, of the magnetic field can be varied by altering the current that is passed through the coil of wire.

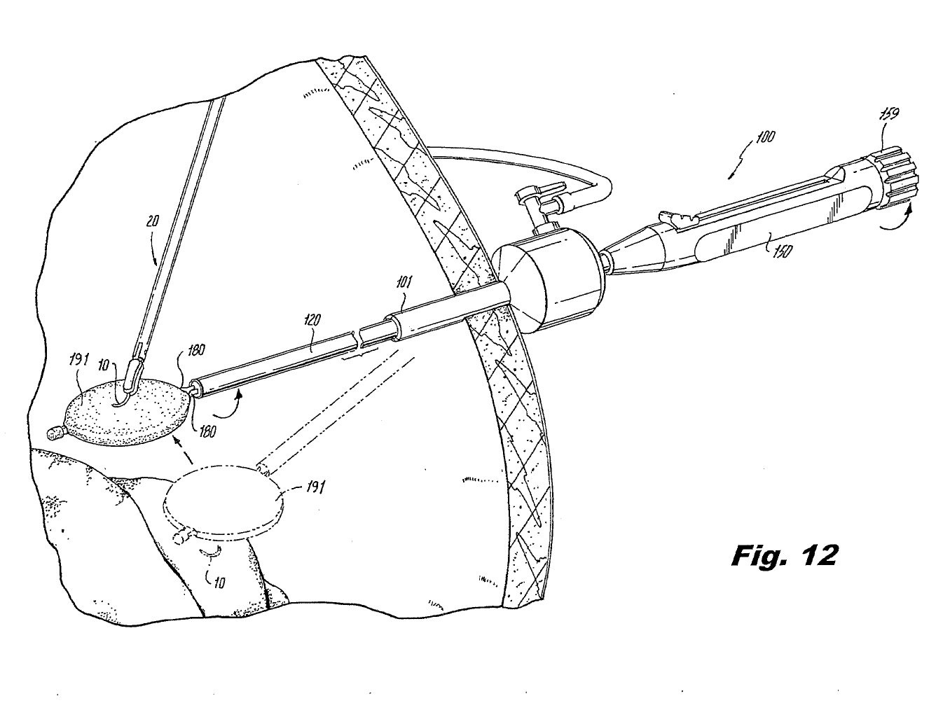

Yet another embodiment and variation of the splines 180 of FIG. 1 is shown in FIG. 12 where a mesh or net-like structure 191 is disposed and coupled to the two splines 180. In the fully collapsed position, the mesh 191 likewise collapses and folds over itself so at to permit the mesh 191 and splines 180 to be retracted within the shaft 120. It will be appreciated that the mesh 191 can either carry one or more permanent magnets or can support one or more electro-magnets (not shown) that are connected to an energy source as by passing wires along or within the splines 180 or by passing wires directly from the inner member 172 to the mesh 191.

The mesh 191 increases the surface area that is available for carrying magnetic elements and for generating a magnetic field having greater coverage. By increasing the scope of the magnetic field, the likelihood of finding the misplaced foreign body increases due to the increased coverage of the magnetic field.

It will be appreciated that in the embodiment of FIG. 12 where a mesh 191 is provided, the splines 180 can include a magnetic element (such as permanent magnets 185) or the splines 180 can be free of the magnetic element 185.

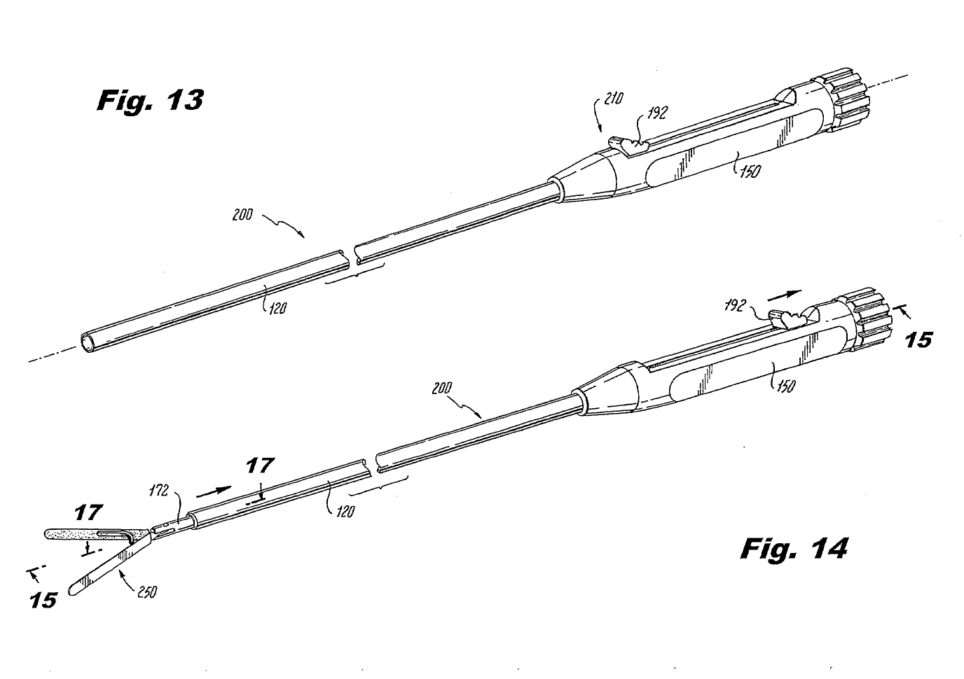

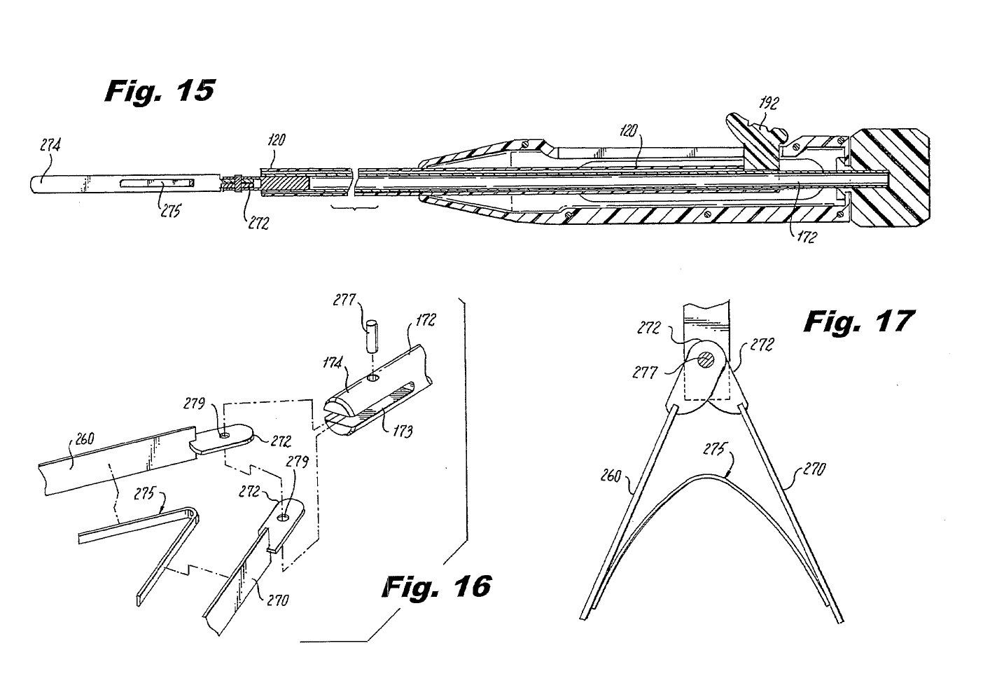

FIGS. 13-17 illustrate a device 200 according to another embodiment. The device 200 is similar to the device 100 and therefore, like elements are numbered alike. The device 200 includes the outer shaft 120 and the inner member 172; however, the actuator means for the device 200 are different in that the tool (e.g., a retrieval mechanism 250) is deployed using a different action than in the device 100. In addition, the tool 250 of the device 200 is different than the tool 170 of device 100.

In the illustrated embodiment, the device 200 includes an actuator 210 that is in the form of a slide mechanism that includes slider 192 that moves within the slot 153 that is formed in the handle 150. Unlike the embodiment of FIG. 1, the slider 192 in FIGS. 13-17 is operatively connected to the outer shaft 120 using a conventional mechanical attachment or linkage. The slider 192 is coupled to the outer shaft 120 such that linear movement of the slider 192 is translated into linear movement of the shaft 120 over and relative to the inner member 172 which in this embodiment is fixedly attached to the handle 150. In other words, in the first embodiment of FIG. 1, the inner member 172 is linearly advanced in a push/pull manner, whereby in the second embodiment of FIG. 13, the outer sheath 120 is the member that is linearly advanced in a push/pull manner so as to expose the tool 250.

The tool 250 has the same intended function as the tool 170; however, the tool 250 has a different construction. In particular, the tool 250 is formed of first and second fingers or blades 260, 270 that are biased open relative to one another by means of a biasing member 275. Each blade 260, 270 has a first end 272 and an opposing second end 274 with the first end 272 being an end that is closer to the inner member 172 and the second end 274 being a distal end of the blade 260, 270. The blades 260, 270 are coupled to the inner member 172 at their first ends 272 in such a way that the blades 260, 270 can pivot open and closed. For example, the first ends 272 of the blades 260, 270 can be received within a slot 273 formed in the inner member 172 at the distal end 174 thereof. A pivot pin 277 passes through the inner member 272 through the slot 273 and likewise passes through holes 279 formed in the blades 260, 270 near the ends 272 thereof. The blades 260, 270 thus pivot open and closed about the pin 277.

Since the blades of the various tools described herein are introduced into the human body and can contact soft tissue, the blades can be treated or configured so as to not adversely impact the tissue if contact is made. For example, at least the edges of the blades 260, 270 can coated or can be formed so that they are smooth or curved so as to not pierce or scratch internal tissue if contact is made therewith. The edges of the blades can thus be rounded or coated with a pliable material that is more giving and can be placed in contact with tissue without adversely impacting the tissue.

The blades 260, 270 can come in any number of different forms so long as when the blades 260, 270 are in a collapsed position, the blades 260, 270 are complementary to one another and can be contained within the outer shaft 120. For example, the two blades 260, 270 in the collapsed position can overlie one another (stacked relationship).

Each blade 260, 270 has a magnetic property in that the blades 260, 270 generate a magnetic field as discussed herein. For example, each blade 260, 270 can be at least substantially formed of a permanent magnet and can be an elongated structure, such an oblong shaped structure or a rectangular shaped structure or even a rod shaped structure. However, the blades 260, 270 can have other shapes so long as they are complementary to one another and can be disposed within the bore of the outer shaft 120 when placed in the fully collapsed position. The blade 260, 270 can thus be formed entirely of a magnetic material.

It will also be appreciated that similar to the blades/splines of the first embodiment, the blade 260, 270 can be formed of a structure that includes a substrate that carries one or more permanent magnets. For example, permanent magnets in the form of one or more strips, pads, discrete shaped magnets, etc., can be provided along a length of the blade 260, 270. As with the previous embodiment, the permanent magnets do not have to extend the entire length of the blade 260, 270 and instead can be located in only one or more discrete sections of the blade 260, 270. For example, a series of circular shaped magnets can be formed along the outwardly facing surface of the blades 260, 270. The permanent magnets can be attached the blades using conventional processing techniques including bonding the magnets to the blades, embedding the magnets within the blade structure, etc.

In this embodiment, the substrates of the blades 260, 270 face one another, with the magnetic components thereof facing outward to generate an outwardly facing magnetic field for locating and retrieving the misplaced foreign body.

The biasing member 275 can be in the form of a spring or the like as shown. The biasing member 275 is coupled to the first blade 260 and the second blade 270 for generating a biasing force that forces the two blades to separate from one another. In the illustrated embodiment, the biasing member 275 can be a bent spring (e.g., V-shaped spring) that is attached at its opposite ends to the two blades 260, 270. In this configuration, the spring 275 can be thought to have two legs with one leg attached to the first blade 260 and the other leg attached to the second blade 270. In the natural, rest position, the legs of the spring 275 flex outwardly and therefore, the blades 260, 270 that are coupled thereto likewise flex outwardly. When the tool 250 is drawn into the shaft 120 or the outer shaft 120 is advanced forwardly over the tool 250, an inward force is applied to the blades 260, 270 to cause the biasing member 275 to flex inwardly and collapse upon itself and store energy. This results in the blades 260, 270 being drawn toward one another toward the fully collapsed position where the blades 260, 270 are disposed adjacent one another and can be received and contained within the outer shaft 120. In the fully collapsed position of the blades 260, 270, the spring 275 contains stored energy that is released as soon as a sufficient amount (length) of the blades 260, 270 is free from the influence of the surrounding outer shaft 120. More specifically, as the tool 250 becomes more and more exposed, the spring 275 begins to release its stored energy to the blades 260, 270 which increasingly have the ability to pivot open. This continues until the tool 250 is completely exposed and extended beyond the outer shaft 120 and all of the stored energy of the spring 275 is released.

It will also be understood that the blades 260, 270 can alternatively be in the form of blades that are non-magnetic in nature; however, an electro-magnetic component can be incorporated into or associated with the blades 260, 270 that act as carriers or substrates (support member). For example, a coil of wire can be wrapped around the blades 260, 270 to form an electro-magnetic element that is operatively connected to an energy source.

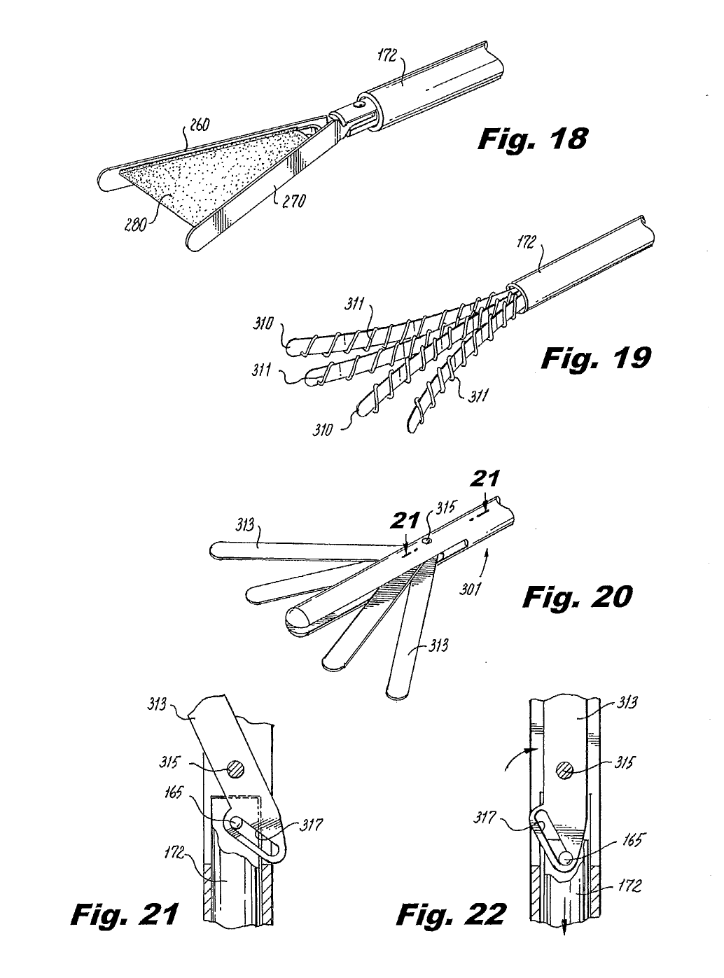

In FIG. 18, a mesh or net-like structure 280 is disposed and coupled to the blades 260, 270. In the fully collapsed position, the mesh 280 likewise collapses and folds over itself so at to permit the mesh 280 and blades 260, 270 to be retracted within the shaft 120. It will be appreciated that the mesh 280 can either carry one or more permanent magnets (not shown) or can support one or more electro-magnets (not shown) that are connected to an energy source as by passing wires along or within the blades 260, 270 or by passing wires directly from the inner member 172 to the mesh 280.

The mesh 280 increases the surface area that is available for carrying magnetic elements and for generating a magnetic field having greater coverage.

It will be appreciated that in the embodiment of FIG. 18 where a mesh 280 is provided, the blades 260, 270 can include a magnetic element (such as permanent magnets) or the blades 260, 270 can be free of a magnetic element.

FIG. 19 illustrate a device 300 that is similar to the devices previously described and therefore like elements are numbered alike. In this embodiment, the distal end of the inner member (shaft) 172 is coupled to one or more flexible blades (fingers) 310. In the illustrated embodiment, there are four blades 310 that are disposed adjacent one another (e.g., stacked orientation).

As with the splines described above with reference to the device 100, the blades 310 are naturally biased to a bent position. More specifically, the blades 310 are designed so that that they naturally move between a fully deployed (extended) position and a fully collapsed (retracted) position. In particular, in a normal, rest position, the blades 310 assume the fully deployed position in which the blades 310 are bent along their lengths. When a force is applied to the blades 310 (e.g., an inwardly directed force), the blades 310 flex inward and this result in the blades 310 straightening out and assuming a more linear form. In other words, as the inward force is applied to the blades 310, the blades 310 move toward the fully collapsed position in which the blades 310 are more linear in shape and can be received within the bore of the shaft 120.

Each blade 310 is constructed so that it has magnetic properties to facilitate locating and retrieving a misplaced foreign body. For example, the blade 310 can have one or more permanent magnets incorporated therein, with the permanent magnets being capable of being provided in different forms, such as strips, pads, discrete magnets, etc. For example, each blade 310 can have at least one magnetic element that is supported by a substrate (blade part) and in particular, the blade 310 can be a layered structure with a flexible substrate 310 that is formed of a material that is outwardly biased and supports the magnetic element. The blades 310 can be formed of the same materials that are suitable for forming the splines. For example, a shape memory alloy can be used to form the blade 310 (at least the substrate part thereof) and impart the desired elastic properties and create the naturally biased position.

It will also be appreciated that, as shown in FIG. 19, each blade 310 can carry and support an electro-magnet 311, such as a coiled wire. More specifically, a wire is coiled around the blade 310 and is operatively connected to an energy source to permit current to pass through the coiled wire to generate the magnetic field. In this manner, the device includes four electro-magnetic elements that create individual magnetic fields and due to the deployment of the blades 310, the scope or extent of the magnetic fields is expanded.

To increase the magnetic field, the blade 310 can be a layered structure that includes the memory alloy and also include a metal layer about which the coiled wire is disposed, thereby increasing the strength of the magnetic field generated by the electro-magnet 311 as described hereinbefore.

FIGS. 20-22 illustrate a device 301 that is similar to device 300 and includes a plurality of blades 313 that are similar to blades 310 and are arranged in a stack relationship. The blades 313 are pivotally attached to the shaft 120 using a pivot 315 that passes through the blades 313. When the blades 313 are deployed, the blades 313 pivot outwardly to produce splayed blades 313. As shown, each blade 313 includes a cam slot 317 that are at least partially aligned with one another when the blades 313 are stacked.

In the illustrated embodiment, the inner member 172 includes a pin 165 that extends outwardly therefrom. The pin 165 is sized to be disposed within and through the cam slots 317 of the stacked blades 313. It will be appreciated that since the blades 313 are fixedly attached to the shaft 120 at the pivot 315, linear movement of the inner member 172 causes the pin 165 to ride within the cam slots 317 and cause pivoting of the blades 313 about the pivot 315. FIGS. 20 and 21 show the blades 313 in a deployed position and FIG. 22 shows the blades 313 in a retracted position.

The actuator mechanism in this device is similar to FIG. 1 in which manipulation of the actuator causes the linear movement of the inner member 172, thereby resulting in the blades 313 pivoting open and closed.

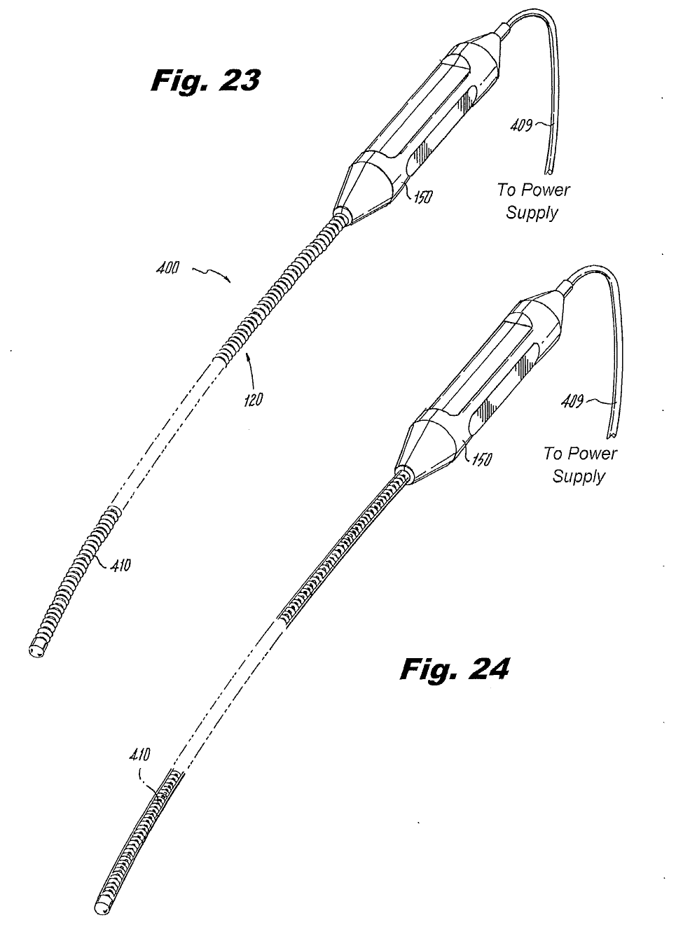

FIGS. 23-24 show a device 400 according to another embodiment that is similar to the other embodiments. As a result, like components are numbered alike. The device 400 is an electro-magnetic based device in which an electro-magnet 410 is disposed along the inner member 172 (FIG. 24). The electro-magnet 410 can be in the form of a coiled wire that is disposed about the inner member 172. The electro-magnet 410 is operatively connected to an energy source to permit the electro-magnet 410 to be energized. For example, the handle of the device 400 can include a plug outlet into which a plug can be plugged or a cord 409 extends therefrom, with the plug being connected to a cord that is attached to an energy device, such as a device that generates an electric current that is delivered to the device. The electro-magnet 410 can be coupled to the plug outlet using conventional means, such as wiring that is routed through the device 400 from the handle to the tool. Circuitry within the external current generating device permits control and the ability to vary the characteristics of the current.

As with the other embodiments, the device 400 (FIG. 24) can be actuated in any number of different ways so to expose the electro-magnet 410. For example, the previously described actuation means can be used including move the outer shaft 120 relative to the inner member 172 to expose the tool or the inner member 172 can be advanced forward relative to the fixed outer shaft 120 so as to expose the tool.

It will further be appreciated that, as shown in FIG. 23, the electro-magnet 410 can be disposed about a fixed member in that the device can simply have the elongated outer shaft 120 which is fixed to the handle 150, with the electro-magnet 410 being disposed about the outer shaft 120 in an exposed position all the time. In this embodiment, the instrument is inserted into the trocar/port and is advanced to the surgical site and then the energy source is delivered to the electro-magnet 410 resulting in the generation of a magnetic field. The user then simply moves the electro-magnet 410 about the surgical site until the misplaced foreign body is within the influence of the magnetic field and thereby is drawn into contact with electro-magnet 410 due to magnetic attraction between the metal-based foreign body and the electro-magnet.

It will also be understood that with all of the electro-mag net based devices described herein, the handle of the respective device can include one or more controls that are specific to the operation of the electro-magnet. For example, the handle can include an on/off button for supplying energy to the coil. It is envisioned that control over the characteristics of the current can be performed by controls that are part of the external device that is the energy source; however, it is within the scope of the present invention, that the handle can include a means for altering the characteristics of the energy (current) that is delivered to the coil.

FIGS. 11 and 12 show the devices of the present invention in use. FIG. 11 shows the device 100 being inserted into the trocar 101 for locating and retrieving a misplaced foreign body 10, such as a needle. Once the retrieval mechanism (tool) 170 is within the body, it is deployed so as to create a magnetic field of greater dimensions due to the deployment of the tool 170. It will be appreciated that the device of the present invention, including device 100, can be used in combination with another tool/instrument 20 that performs the actual removal of the misplaced foreign body 10 from the body cavity. More specifically, once the device 100 of the present invention successfully locates and is coupled to the misplaced foreign body 10 as by magnetic attraction, another instrument, such as a gripping device, can be introduced to the site (as by another port) and can be moved into position to grasp and remove the misplaced foreign body 10.

It will be appreciated that the devices discloses herein are intended for use with conventional equipment that is used in a minimally invasive surgical procedure and therefore, the device in the collapsed position is sized and shaped to permit it to be received within conventional trocars/ports used during the procedure. The device, in the collapsed position, can have a width of about 10 mm or less. Other dimensions are possible and may be preferred depending upon the particular application that the device is being used in; however, the devices of the present invention are particularly suited for use in a minimally invasive surgical environment.

It will also be further appreciated that the internal mechanism for causing the retrieval tool to move between the deployed position and the collapsed position can be any number of different mechanical attachment or linkage mechanism, including those described in U.S. Pat. Nos. 5,195,505; 5,199,419; 5,271,385; 5,381,788; 5,391,180; 5,490,819; 5,414,517; 5,554,101; 5,607,450; and 5,766,205, each of which is hereby incorporated by reference in its entirety.

In yet another embodiment, the deployable member that is part of the retrieval tool and that carries the magnetic element can be in the form of a single blade structure as opposed to the multiple blade structures described herein. In this embodiment, the single blade can be deployed and when deployed will change its shape compared to the collapsed position. For example, the blade can be formed of a shape memory alloy, etc., as described herein, and will curl or bend when deployed. In addition, the blade can be a single blade structure but when deployed can occupy a greater area. For example, the blade can be formed to have a bellows type structure (fan-fold design) and when deployed, the bellows structure unfolds and therefore, the magnetic elements that are associated with the bellows structure unfold and/or are spaced over a greater area, thereby generating a magnetic field that occupies a greater coverage area.

In accordance with the present invention, the various retrieval tools described herein are in some way manipulated to cause a change in at least one characteristic or property of the tool which results in a magnetic field being generated and/or being generated over a greater area. For example, one type of manipulation is the activation of an electro-magnetic device to cause a current to flow through the device, thereby generating the magnetic field. Yet another type of manipulation is the deployment or altering of the physical characteristics of the magnetic element such that the generated magnetic field extends over a greater area. This action can either be the bending, upon deployment, of one or more fingers that carry the magnetic elements as described or it can be another action, such as the above-described unfolding of a fan-fold or bellows type structure that results in the magnetic elements occupying a greater footprint and thereby, generating a magnetic field that has greater coverage.

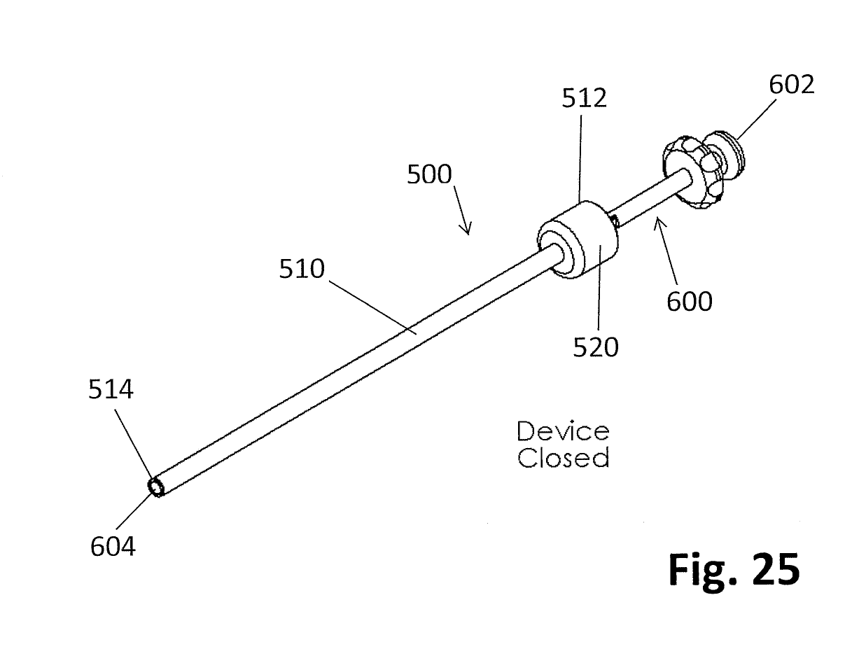

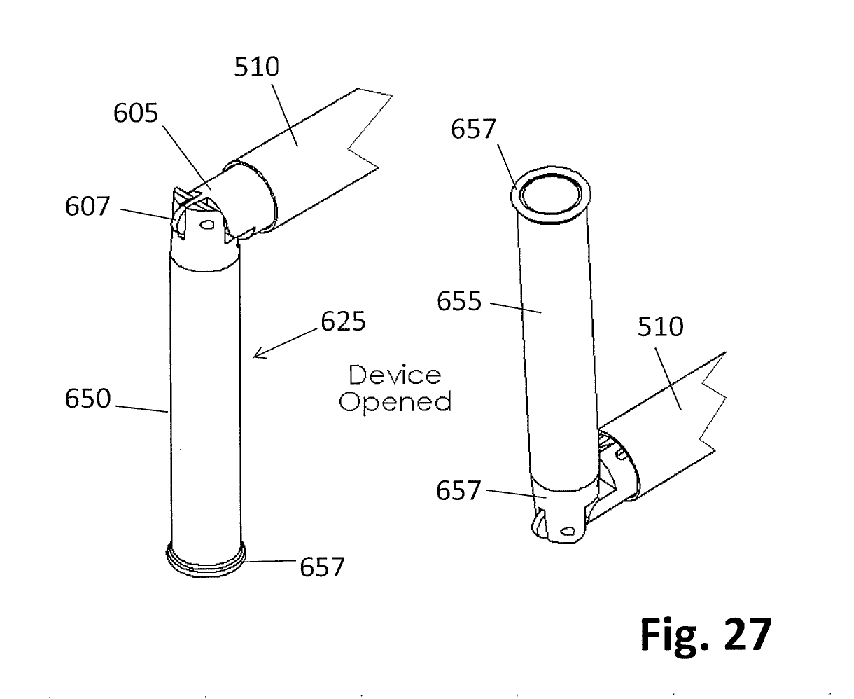

FIGS. 25-31 illustrate a device 500 according to another embodiment for insertion into a conventional trocar/port for locating and retrieving a misplaced magnetic foreign body. The device 500 is similar to the other devices disclosed in the related '814 application and is designed to perform the same functions described therein.

The device 500 can include an outer sheath or shaft 510 that is open at both proximal and distal ends 512, 514, respectively, and thus represents a hollow structure. As shown, the proximal end 512 can include an enlarged body or handle member 520. The illustrated outer sheath 510 is in the form of a tubular structure and the length and shape thereof can be selected depending upon the particular application. However, the dimensions of the outer sheath 510 are selected in view of the trocar dimensions since the outer sheath 510 is inserted into and passes through the hollow bore of the trocar. In one embodiment, the outer sheath 510 has a diameter of about 12 mm; however, other dimensions are possible depending in part on at least the dimensions of the trocar. In addition, other factors can influence selection of the outer sheath 510.

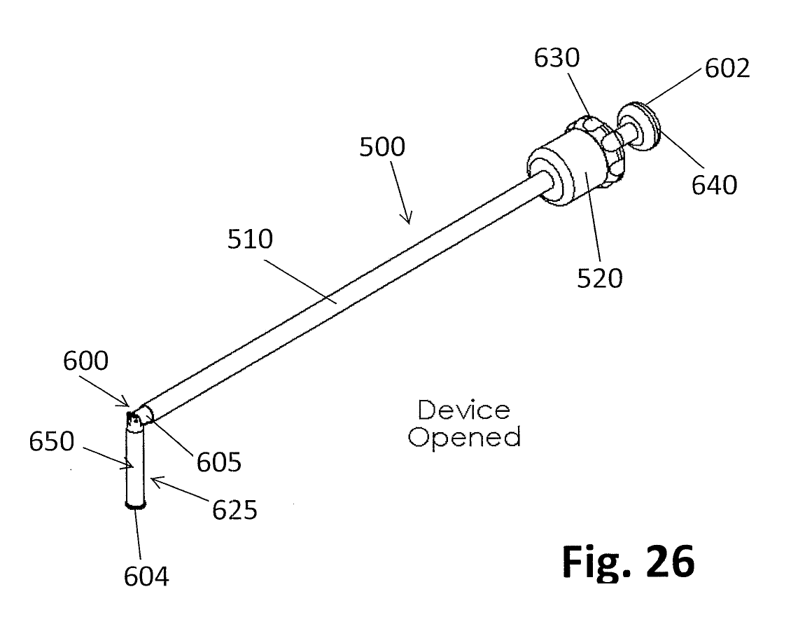

The device 500 further includes an inner member 600 which is designed to slidingly move within the outer sheath 510 and be positioned between an extended (opened) position (FIG. 26) in which a distal end 604 of the member 600 extends beyond the distal end 514 of the outer sheath 510 and a retracted (closed) position in which the distal end 604 of the member 600 is at least partially retracted within the outer sheath 510. In the retracted (closed) position, the distal end 604 of the member 600 can be fully contained within the outer sheath 510.

The dimensions of the inner member 600 are thus such that it can be received within the hollow bore of the outer sheath 510.

The inner member 600 also includes a proximal end 602 which can include a handle component 630 and an actuator 640 which causes controlled movement of the inner member 600 as described below. The handle component 630 can be a knob-like structure that has ribs to assist the user in grasping the inner member 600 and manipulating the inner member 600 relative to the outer sheath 510. For example, the user can rotate the inner member 600 within and relative to the outer sheath 510 by rotating the handle component 630. The handle component 630 has a greater size than the member 520 and thus the member 520 serves as a stop that limits the longitudinal movement of the inner member 600 within the hollow bore of the outer sheath 510. For example and as illustrated, in the closed (retracted) position of FIG. 25, the handle component 630 is spaced from the member 520, while in the opened (extended) position of FIG. 26, the handle component 630 is in contact with or proximate to the member 520. The handle component 630 is thus fixed to the elongated body that defines the inner member 600.

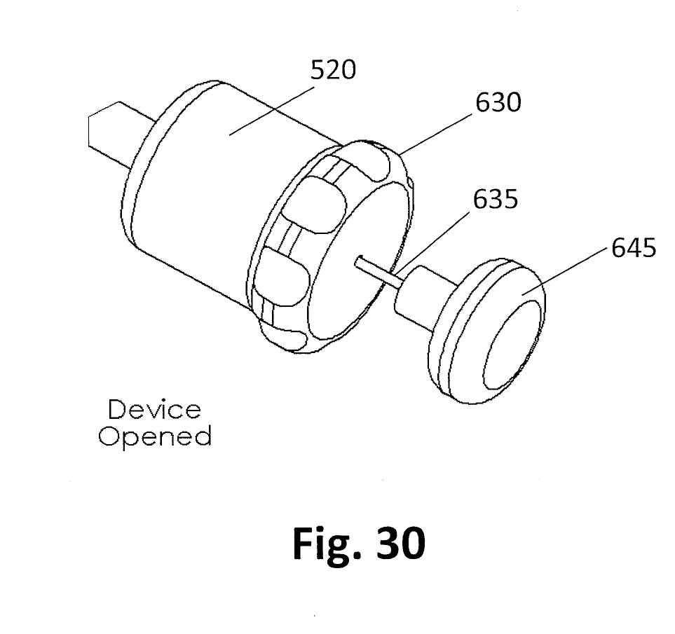

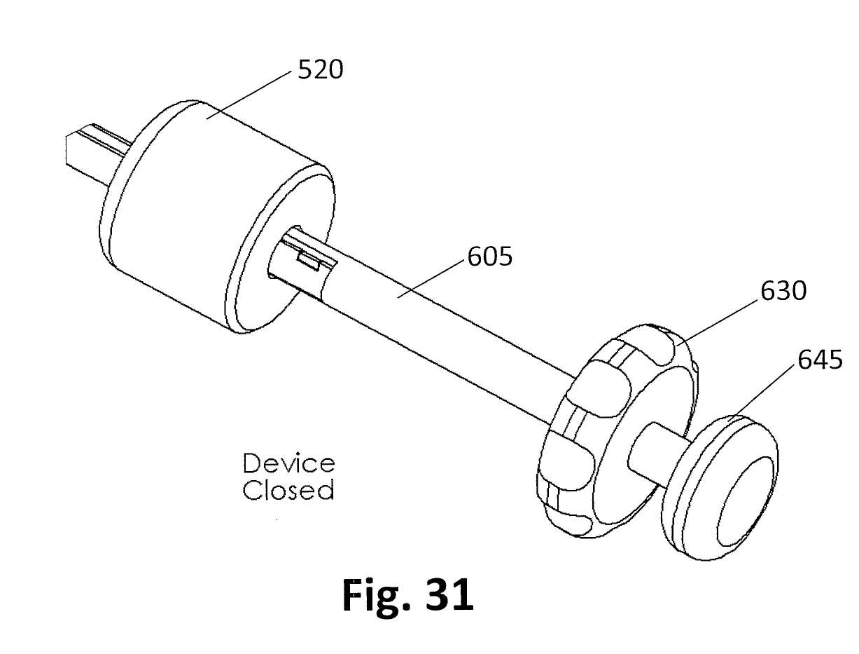

The actuator 640 is shown in FIGS. 30 and 31 and is configured to permit at least a portion 625 of the distal end 604 of the inner member 600 to move (e.g., to pivot) as described below. The actuator 640 can take any number of different forms including a lever or pull member (pull knob) as shown which is coupled to a mechanical linkage that is coupled to the movable distal end portion 625. The actuator 640 is thus intended to allow the user to easily cause controlled movement of the distal end portion 625. It will be appreciated and better understood in view of the below discussion that the illustrated actuator 640 is part of a pull wire mechanism in which a pull wire 635 is coupled to a pull knob 645 at the proximal end and at the distal end is coupled to the distal end portion 625.

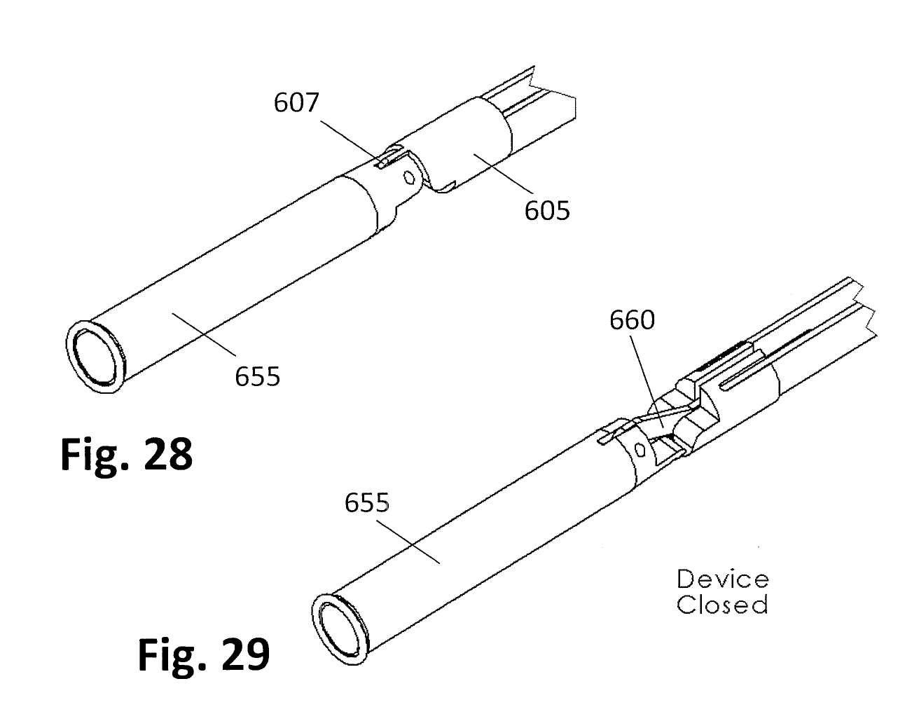

As shown best in FIGS. 28 and 29, the inner member 600 is an elongated structure that includes a main portion 605 and the distal end portion 625 which is coupled to a distal end 607 of the main portion 605.

The distal end portion 625 of the inner member 600 which is controllable is in the form of an articulating member and in particular, an articulating magnetic element 650 similar to the other embodiments described in the '814 application. The coupling between the magnetic element 650 and the distal end 607 of the main portion 605 can be accomplished using any number of different types of mechanical couplings. In the illustrated embodiment, the magnetic element 650 is attached to the main portion 605 using a mechanical link 660 that allows the magnetic element 650 to pivot relative to the main portion 605.

The pull wire 635 is attached to the link 660 and the proximal end of the magnetic element 650 is pivotally coupled to the distal end of the main portion 605 about the link 660 such that translational movement of the pull wire 635 causes a pivoting of the magnetic element 650. As shown in FIG. 30, in the open position (extend position) in which the magnetic element 650 is pivoted, the pull knob 645 is pulled from the handle component 630 and the pull wire 635 is exposed. FIG. 31 shows the closed (retracted) position in which the magnetic element 650 is not pivoted but instead lies linearly along the axis of the main portion 605 and as shown, the pull knob 645 is in contact with the handle component 630 and the pull wire 635 is not visible.

In one embodiment, the magnetic element 650 pivots over a range of 90 degrees; however, in other embodiments, the magnetic element 650 can pivot over a range greater than 90 degrees. However, since the inner member 600 is freely rotatable, a 90 degree pivot range is generally sufficient.

It will be understood that any number of other mechanical linkages can be used to caused the controlled movement of the magnetic element 650 besides the illustrated linkage. In particular, the user simply needs to be able to control the magnetic element from a location outside the body and be able to easily use a slider or control knob or some other actuator to cause the controlled movement (pivoting) of the magnetic element.

The magnetic element 650 includes a magnetic component 655 and can include a casing or housing or end caps 657, etc. As with the other embodiment in the '814 application, the strength of the magnetic component 650 should be such that magnetic foreign material within a body is attracted to and drawn into contact with the magnetic component 650. The magnetic component 650 can be in the form of a permanent magnet. For example, one type of permanent magnet that is particularly suited for the present invention and the intended applications of the device 500 is a neodymium magnet (also known as NdFeB, NIB, or Neo magnet), the most widely-used type of rare-earth magnet, is a permanent magnet made from an alloy of neodymium, iron, and boron to form the Nd₂Fe₁₄B tetragonal crystalline structure. As is known in the industry, the magnetic strength (field) is in part based on the characteristics of the magnet, such as its diameter (width) and length. The greater the diameter, the stronger the magnetic field and similarly, the longer the magnet, the stronger the magnetic field.

In one embodiment, the magnetic component 655 includes a neodymium magnet having a diameter of about ⅜ inch and a length of about 2 inch. It will be appreciated that other dimensions are equally possible for the magnet depending upon the intended application and the overall design and dimensions of the device. It will be appreciated that the width of the magnet cannot exceed the width of the inner bore formed in the outer sheath 510 and obviously is less than the width of the trocar through which the device 500 is fed.

Preferably, the strength of the magnet is such that magnetic material that is at least 1 inch away or greater is attracted to and drawn into contact with the magnet and more preferably, material that is at least 2 inches away or greater is attracted to and drawn into contact with the magnet. This objective and the above field of attraction distances relate to foreign magnetic material that may be accidentally misplaced in the body during the course of a surgery as discussed in the '814 application.

In order to permit coupling to the link 660, a proximal cap or housing is provided and is attached to the magnet itself using conventional means, including bonding, etc. A distal end protective cap can also be provided as illustrated. The proximal part is the portion that is pivotally connected to the main portion 605 using the link or some other mechanical attachment such as a pin, etc.

It will also be appreciated that the mechanical linkage that is operatively coupled to the magnetic element 650 can include one or more locking positions to allow the user to lock the magnetic element 650 in any one of several pivoted positions. For example, the magnetic element 650 can be locked in a 45 degree position; a 90 degree position, etc. Such lock can lock the mechanical linkage in a fixed position, thereby preventing the accidental movement of the magnetic element 650.

It will also be appreciated that the device 500 can consist solely of the inner member 600 and in that case, the outer sheath 510 is eliminated and the inner member 600 is the only member that is inserted and passed through the inner bore of the trocar.

The operation and use of the device 500 are described below.

As with the other embodiments described in the '814 application, the device 500 is inserted into and passed through the trocar until at least the distal tip of the instrument extends beyond the trocar distal tip within the patient's body. When the device 500 include outer sheath 510, the user then manipulates and directs the inner member 600 forward toward the patient to cause at least the magnetic element 650 to extend beyond the distal end of the sheath 510. In this initial position as shown in FIGS. 28 and 29, the magnetic element 650 is in-line (axially aligned) with the main portion 605. Since the magnetic element 650 has cleared the outer sheath 510, it can be manipulated by the user and this includes the ability to fully rotate the magnetic element 360 degrees and the ability to move (pivot) the magnetic element 650 so that it is out of the plane of the main portion 605. As discussed hereinbefore, the magnetic element 650 can be pivoted into different position so as to allow the user to position the magnet and thus the associated magnetic field in different positions so as to attract the magnetic foreign material that is to be located and recovered by the device 500.

Further aspects of the misplaced foreign material and the patient's anatomy and potential applications for the device 500 are set forth in the '814 application and therefore are not repeated herein. It will therefore be understood that the device 500 is in intended to be used in the manner described and in all of the applications described in the '814 application. Also, a retrieval tool, such as grippers or the like can be used in combination with the device 500 to actually remove the misplaced foreign material once it has been located and retrieved by the device 500.

As mentioned above, when the outer sheath 510 is eliminated, the inner member 600 is passed through the trocar and then manipulated in the same manner described above.

While the invention has been described in connection with certain embodiments thereof, the invention is capable of being practiced in other forms and using other materials and structures. Accordingly, the invention is defined by the recitations in the claims appended hereto and equivalents thereof.

What is claimed is:

1. A device for locating and retrieving a misplaced foreign body that has metallic characteristics comprising:
a hollow shaft connected to a handle and including a distal end; and
a retrieval tool that is at least partially received within an interior of the hollow shaft, the retrieval tool having a movable tip portion that includes a magnetic element that generates a magnetic field, the movable tip being operatively coupled to a mechanism that is configured to move the tip portion between at least a first position and a different second position, the retrieval tool being slidable movable within the hollow shaft so as to permit the tip portion to extend beyond the distal end of the hollow shaft, the movable tip being movable to the second position only when the movable tip is completely beyond the distal end of the hollow shaft,
wherein an angle is formed between the tip portion in the second position relative to the first position;
wherein the magnetic element is in the form of an elongated magnet.

2. The device of claim 1, wherein in the first position, the tip portion is axially aligned with an adjacent main portion of the retrieval tool and is position to allow the tip portion to be received within the interior of the hollow shaft.

3. The device of claim 1, wherein the tip portion is pivotable between the first and second positions.

4. The device of claim 1, wherein the mechanism is configured such that linear movement of a linkage is translated into a pivoting action of the tip portion.

5. The device of claim 1, wherein the angle between the tip portion in the first and second positions is between 0 and 90 degrees.

6. The device of claim 1, wherein the magnetic element comprises a permanent neodymium magnet.

7. The device of claim 6, wherein the neodymium magnet has an elongated rod shape and has a diameter of at least about ⅜ inch and a length of at least about 2 inch.

8. The device of claim 1, wherein the retrieval tool has an actuator at a proximal end that is operatively coupled to the mechanism to allow user manipulation which is translated into a pivoting of the tip portion, the actuator being oversized to prevent reception thereof into the interior of the hollow shaft.

9. The device of claim 1, wherein the first position, the shaft and retrieval tool have a width of about 10 mm or less to allow the device to be used in ports associated with minimally invasive surgery.

10. A device for locating and retrieving a misplaced foreign body that has metallic characteristics comprising:
a shaft connected to a handle; and
a retrieval tool that is coupled to the shaft and moves between a deployed position and collapsed position, the tool having a magnetic element that generates a magnetic field, wherein the magnetic field extends over a greater area in the deployed position and is of sufficient strength to attract the foreign body such that it is held in contact with the tool.

11. The device of claim 1, wherein the retrieval tool comprises at least two blades that are biased outward in the deployed position and assume a substantially parallel relationship in the collapsed position, each blade carrying at least one permanent magnet.

12. The device of claim 2, wherein each blade is formed of a shape memory alloy that is naturally biased outward to assume a curved configuration.

13. The device of claim 2, wherein the shaft includes an outer tube and an inner member that is disposed within the outer tube and can linearly travel therein, the retrieval tool being coupled to the inner member such that when the retrieval tool is exposed beyond a distal end of the outer tube, the blades automatically are biased outward and assume the deployed position where the blades are curved, the blades being in the collapsed position when the blades are contained within the outer tube.

14. The device of claim 4, wherein the handle is fixedly attached to the outer tube and includes an actuator that is coupled to the inner member and movement of the actuator causes the inner member to move linearly relative to the fixed outer tube to cause the retrieval tool to move between the deployed and collapsed positions.

15. The device of claim 4, wherein the handle is fixedly attached to the inner member and includes an actuator that is coupled to the outer tube and movement of the actuator causes the outer tube to move linearly relative to the fixed inner member to cause the retrieval tool to move between the deployed and collapsed positions.

16. The device of claim 1, wherein the retrieval tool comprises at least two blades that are biased outward in the deployed position and assume a substantially parallel relationship in the collapsed position, each blade carrying at least one electro-magnet element that when connected to a source of electric current emits a magnetic field.

17. The device of claim 2, wherein ends of the blades are pivotally attached to an end of the inner member and a biasing member is disposed between the blades such that when the blades are disposed beyond the outer tube, the biasing member applies an outward biasing force to cause splaying of the blades to the deployed position.

18. The device of claim 8, wherein each blade comprises a permanent magnet.

19. The device of claim 8, wherein each blade includes a substrate that supports the at least one permanent magnet.

20. The device of claim 8, wherein each blade carries at least one electro-magnet element that when connected to a source of electric current emits a magnetic field.

21. The device of claim 4, wherein the inner member is rotatable within the outer tube to permit rotation of the tool beyond the outer tube.

22. The device of claim 4, wherein the blades are arranged in a stacked relationship and are pivotally attached to the inner member about a common pivot, each blade including a cam slot proximate one end of the blade proximate the pivot, the inner member including a cam pin that passes through the cam slots of the stacked blades, wherein linear movement of the inner member within the outer tube causes movement of the pin within the cam slots which are shaped and located such that movement of the pin within the cam slots causes the blades to move between the deployed and collapses positions.

23. The device of claim 1, wherein, in the collapsed position, the shaft and retrieval tool have a width of about 10 mm or less to allow the device to be used in ports associated with minimally invasive surgery.

24. The device of claim 1, wherein the foreign body comprises a needle or metal surgical instrument.

25. A device for locating and retrieving a misplaced foreign body that has metallic characteristics comprising:
a shaft connected to a handle;
an electro-magnetic element that is disposed about the shaft and is configured to generate a magnetic field when electric current passes therethrough, wherein the shaft and electro-magnetic element have a width of about 10 mm or less to allow the device to be used in ports associated with minimally invasive surgery.

26. The device of claim 16, wherein the electro-magnetic element comprises a wire coiled about the shaft and the handle has a power cord attached thereto that carries a conductive element that is operatively attached to the coiled wire.

27. A method for locating and retrieving a misplaced metal foreign body within a cavity of the body comprising the steps of:
inserting a port through an incision made in the body, the port providing a conduit to the cavity;
inserting a device through the conduit of the port for locating and retrieving the misplaced metal foreign body, the device including a shaft connected to a handle and a retrieval tool that is coupled to the shaft and moves between a deployed position and collapsed position, the tool having a magnetic element that generates a magnetic field;
deploying the retrieval tool after the retrieval tool has cleared the port and is within the cavity, wherein the magnetic field extends over a greater area in the deployed position; and
moving the retrieval tool within the cavity until the misplaced foreign body is under the influence of the magnetic field and is drawn into contact and held against the magnetic element of the retrieval tool.

28. The method of claim 18, wherein the magnetic element comprises a permanent magnet.

29. The method of claim 18, wherein the magnetic element comprises an electro-magnetic element.

30. The method of claim 18, wherein the retrieval element includes at least two blades that are biased outward in the deployed position, due to the blades having a curved shape in a rest position and the step of deploying the retrieval tool comprises exposing the retrieval element beyond the shaft so as to cause the blades to naturally bias outwardly and assume the deployed position.
